(19) United States
(12) Patent Application Publication
(10) Pub. No.: US 2026/0357170 A1
(43) Pub. Date: Nov. 12, 2026

(54) FLASK TRAY, FLASK UNIT, AND CELL CULTURE SYSTEM

(71) Applicant: MITSUBISHI HEAVY INDUSTRIES, LTD., Tokyo (JP)

(72) Inventors: Shinichi OKAMOTO, Tokyo (JP); Takashi MATSUZAWA, Tokyo (JP); Hirone KOMATSU, Tokyo (JP); Naoki OGAWA, Tokyo (JP); Akihisa ENDO, Tokyo (JP); Yuki ASAI, Tokyo (JP); Yuhei HIBINO, Kitakyushu-shi (JP); Shigehiro SUGIYAMA, Tokyo (JP); Nobuyasu SAKATA, Tokyo (JP); Tomohiro HARA, Tokyo (JP); Takehisa OKUDA, Tokyo (JP); Takafumi SHINOGI, Tokyo (JP)

(73) Assignee: MITSUBISHI HEAVY INDUSTRIES, LTD., Tokyo (JP)

(21) Appl. No.: 19/487,975

(22) PCT Filed: Feb. 8, 2024
(86) PCT No.: PCT/JP2024/004411

§ 371 Date: Nov. 25, 2025

(30) Foreign Application Priority Data
Jul. 5, 2023 (JP) 2023-110874

(51) Int. Cl. C12M 1/12; C12M 1/00; C12M 3/00

(52) U.S. Cl. CPC C12M 23/04; C12M 23/38; C12M 23/48

(57) ABSTRACT

A flask tray includes: a placing plate having a placing surface on which a bottom surface of a flask is placed; a support portion that extends upward from the placing plate; and a frame that is supported by the support portion above the placing plate and surrounds the flask from a horizontal direction such that the flask can be placed on the placing surface from above, in which the frame has a lid placing portion that is disposed adjacent to the flask and that is capable of holding a lid portion that is detachably attached to the flask.

### TECHNICAL FIELD

The present disclosure relates to a flask tray, a flask unit, and a cell culture system.

Priority is claimed on Japanese Patent Application No. 2023-110874, filed Jul. 5, 2023, the content of which is incorporated herein by reference.

### BACKGROUND ART

Patent Document 1 discloses a culture container set having a recessed portion capable of holding a plurality of case-shaped culture containers in which a culture solution is accommodated.

### CITATION LIST

### Patent Document

Patent Document 1: Japanese Patent No. 4403169

### SUMMARY OF INVENTION

### Technical Problem

By the way, in a case of performing cell culture, a flask with a lid may be used as a container for accommodating a culture solution. In a case where such a flask with a lid is handled in a cell culture system, handling of the lid in a case of performing each operation on the culture solution is a problem.

That is, for example, after a dispensing operation is performed on the culture solution in the flask, it is necessary to attach the lid portion again on the spot. Therefore, the flask on which the next dispensing operation is to be performed needs to wait for the time required for the attachment of the lid, and thus the operation efficiency is lowered.

In addition, in a case where the removed lid portion is attached to another flask, contamination between the culture solutions occurs, which is not preferable.

The present disclosure provides a flask tray, a flask unit, and a cell culture system, in which it is possible to improve operation efficiency and to suppress occurrence of contamination.

### Solution to Problem

A flask tray according to the present disclosure includes: a placing plate having a placing surface on which a bottom surface of a flask having a lid portion that is attachable to and detachable from the flask is placed; a support portion that extends upward from the placing plate; and a frame that is supported by the support portion above the placing plate and that surrounds the flask from a horizontal direction such that the flask is capable of being placed on the placing surface from above, in which the frame has a lid placing portion that is disposed adjacent to the flask and that is capable of bolding the lid portion.

A flask unit according to the present disclosure includes: the flask tray described above; and the flask having the lid portion.

A cell culture system according to the present disclosure includes: the flask unit described above; an incubator that forms a culture area in which the flask unit is accommodated; a clean bench that forms a liquid operation area for performing a dispensing operation on the flask unit; a transport device that is capable of transporting the flask unit in each of the culture area and the liquid operation area; and a pass box that is capable of transferring the flask unit between the incubator and the clean bench.

### Advantageous Effects of Invention

According to the flask tray, the flask unit, and the cell culture system of the present disclosure, it is possible to improve treatment efficiency and to suppress occurrence of contamination.

### BRIEF DESCRIPTION OF DRAWINGS

FIG. 1 A schematic plan view showing the schematic configuration of a cell culture system according to a first embodiment of the present disclosure.

FIG. 2 A perspective view of a flask unit of the cell culture system according to the first embodiment of the present disclosure.

FIG. 3 A plan view of the flask unit of the cell culture system according to the first embodiment of the present disclosure.

FIG. 4 A front view of the flask unit of the cell culture system according to the first embodiment of the present disclosure.

FIG. 5 A side view of the flask unit of the cell culture system according to the first embodiment of the present disclosure.

FIG. 6 A longitudinal sectional view of a recessed portion of a flask tray in the flask unit of the cell culture system according to the first embodiment of the present disclosure.

FIG. 7 A longitudinal sectional view of a recessed portion of a flask tray in a flask unit of a cell culture system according to a second embodiment of the present disclosure.

FIG. 8 A side view of a flask unit of a cell culture system according to a third embodiment of the present disclosure.

FIG. 9 A perspective view of a flask unit of a cell culture system according to a fourth embodiment of the present disclosure.

FIG. 10 A plan view of a flask unit of a cell culture system according to a first modification example of the embodiment of the present disclosure.

FIG. 11 A plan view of a flask unit of a cell culture system according to a second modification example of the embodiment of the present disclosure.

### DESCRIPTION OF EMBODIMENTS

### First Embodiment

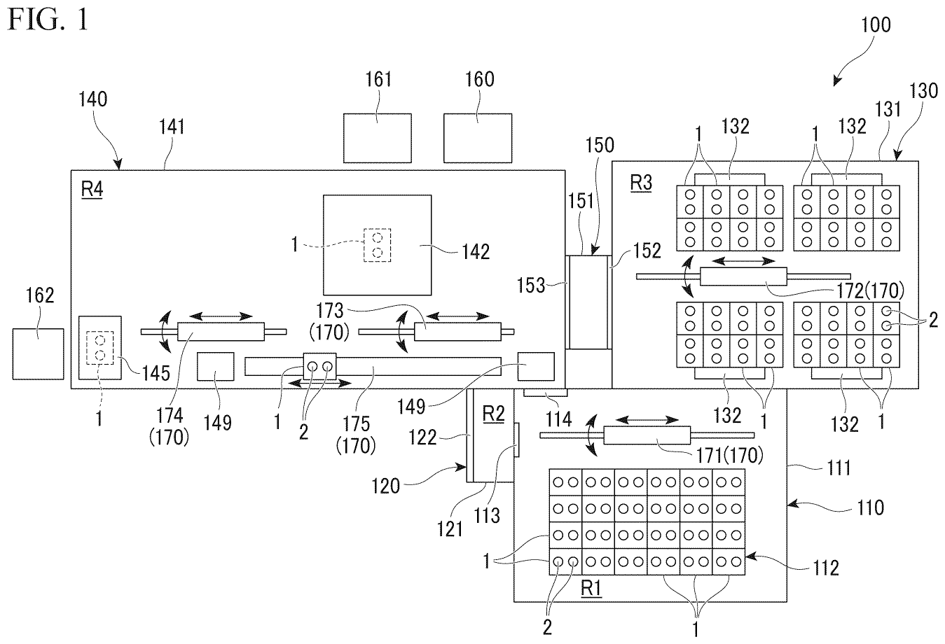

Hereinafter, a first embodiment of the present disclosure will be described in detail with reference to the drawings. As shown in FIG. 1, a cell culture system 100 of the present embodiment is a system that performs cell culture, analysis of cells after culture, or the like by using a flask unit 1 as a treatment unit, and these treatments are performed, for example, in an unmanned and automatic manner.

The flask unit 1 includes a plurality of (in the present embodiment, for example, two) flasks 2. The flask 2 accommodates cells and a culture solution containing a culture medium to which nutrients of the like necessary for cell proliferation are added.

### Cell Culture System

As shown in FIG. 1, the cell culture system 100 includes a stocker 110, an incubator 130, a clean bench 140, and a pass box 150. The cell culture system 100 according to the present embodiment further includes a loading booth 120, a measurement device 160, a culture medium supply device 161, a waste liquid tank 162, and a transport device 170.

### Stocker

The stocker 110 is a facility for storing a plurality of the flask units 1. The stocker 110 includes a stocker main body 111, a rack 112, a stocker first door 113, and a stocker second door 114.

The stocker main body 111 is a housing that forms a storage area R1 as a warehouse inside. The stocker main body 111 is configured with a transparent member such as acrylic or glass.

The rack 112 is disposed in the storage area R1, and is capable of storing the plurality of flask units 1 in a horizontal direction and an up-down direction.

The stocker first door 113 is a door for loading the flask unit 1 or the like from the outside, and is capable of being opened and closed, for example, by sliding in the horizontal direction.

The stocker second door 114 is a door for unloading the flask unit 1 to the clean bench 140, and is capable of being opened and closed, for example, by sliding in the horizontal direction.

### Loading Booth

The loading booth 120 is a booth that is used for loading the flask unit 1 or the like into the stocker 110 from the outside. The leading booth 120 has a booth main body 121 and a loading door 122.

The booth main body 121 is a housing that is disposed adjacent to the stocker 110, and is configured with a transparent member. A space in the loading booth 120 is a loading area R2, and is capable of communicating with the storage area R1 via the stocker second door 114.

The loading door 122 is a door for loading the flask unit 1 or the like from the outside into the loading booth 120, and is capable of being opened and closed, for example, by sliding up and down. The inside of the loading area R2 is managed to have, for example, a negative pressure compared to the atmosphere of the storage area R1. In this way, the entrance of the outside air or the like into the stocker 110 when the loading door 122 is in an opened state is avoided.

### Incubator

The incubator 130 is a facility for proliferating cells in a culture solution. The incubator 130 includes an incubator main body 131 and a shaking stage 132.

The incubator main body 131 is a housing that forms a culture area R3 inside. The culture area R3 is managed to have a temperature, humidity, and carbon dioxide concentration suitable for cell culture. The incubator main body 131 is configured with, for example, a transparent member such as acrylic or glass.

The shaking stage 132 is a device for shaking the flask unit 1. A plurality of the shaking stages 132 are disposed in the culture area R3, and each of the shaking stages 132 holds the plurality of flask units 1. The shaking stage 132 is configured to be able to shake the flask unit 1, for example, by performing eccentric rotation about a vertical axis.

### Clean Bench

The clean bench 140 is a facility for performing various operations and treatments on the flask unit 1. The clean bench 140 includes a clean bench main body 141, a dispensing device 142, a waste liquid extracting device 145, and a stage 149.

The clean bench main body 141 is a housing that forms a liquid operation area R4 which is a space (bio-clean room) having a high cleanliness and being in a sterile state. In the clean space, air flows and circulates as a down-flow (descending air current) from an upper side to a lower side. The clean bench main body 141 is configured with, for example, a transparent member such as acrylic or glass.

The dispensing device 142 is provided in the clean room. The dispensing device 142 performs various dispensing operations and other treatment on the flask unit 1. It should be noted that a plurality of the dispensing devices 142 may be provided instead of one.

The waste liquid extracting device 145 is provided in the liquid operation area R4, and is a device for extracting a culture solution that is not used from the flask 2 of the flask unit 1 as a waste liquid.

A plurality of the stages 149 are provided in the liquid operation area R4. The flask unit 1 is temporarily placed on the stage 149.

### Pass Box

The pass box 150 is provided between the incubator 130 and the clean bench 140. The pass box 150 moves the flask unit 1 between the incubator 130 and the clean bench 140. The pass box 150 includes a box main body 151, a first door 152, and a second door 153.

The first door 152 and the second door 153 are not simultaneously opened. In this way, the culture area R3 and the liquid operation area R4 do not directly communicate with each other. The transfer of the flask unit 1 between the culture area R3 and the liquid operation area R4 is performed through the pass box 150.

The box main body 151 is a housing formed to be interposed between the incubator main body 131 and the clean bench main body 141.

The first door 152 is opened and closed, for example, in the horizontal direction to switch between a communication state and a non-blocking state between the space in the box main body 151 and the culture area R3. The second door 153 is opened and closed, for example, in the horizontal direction to switch between a communication state and a non-communication state between the space in the box main body 151 and the liquid operation area R4.

The first door 152 and the second door 153 are not simultaneously opened. In this way, the culture area R3 and the liquid operation area R4 do not directly communicate with each other

### Measurement Device

The measurement device 160 is disposed adjacent to the clean bench 140. The measurement device 160 is a device capable of performing measurement or analysis of the number of cells in the culture solution. The culture solution dispensed front the flask 2 by the dispensing device 142 is transferred to the measurement device 160 via a tube (not shown).

### Culture Medium Supply Device

The culture medium supply device 161 is disposed adjacent to the clean bench 140. The culture medium supply device 161 is a device capable of supplying a culture medium that is to be accommodated in the flask 2. The culture medium supplied from the culture medium supply device 161 is transferred to the dispensing device 142, and is supplied into the flask of the flask unit 1 in the dispensing device 142.

### Waste Liquid Tank

The waste liquid tank 162 is disposed adjacent to the clean bench 140. The waste liquid tank 162 is connected to the waste liquid extracting device 145 by a tube (not shown). The waste liquid from the waste liquid extracting device 145 is transferred to the waste liquid tank 162 via the tube.

### Transport Device

The transport device 170 is a device for realizing full automation of the transport of the flask unit 1 in the cell culture system 100. The transport device 170 includes a first transport robot 171, a second transport robot 172, a third transport robot 173, a fourth transport robot 174, and a conveyor 175.

### Operation of Cell Culture System

In the cell culture system 100 having the configuration as described above, the culture solution of the flask unit 1 is shaken in the incubator 130, so that cell proliferation is promoted. The flask unit 1 in which the cell proliferation has progressed is transferred to the liquid operation area R4 via the second transport robot 172 and the pass box 150. The flask unit 1 transferred to the liquid operation area R4 is transferred to the dispensing device 142 via the third transport robot 173. In the dispensing device 142, the culture solution is dispensed from the inside of the flask of the flask unit 1, and the culture solution is transferred to the measurement device 160. In the measurement device 160, the number of cells in the culture solution, the analysis of the liquid properties, and the like are performed.

In addition, when the culture medium is added to the flask 2, the flask 2 is transported into the dispensing device 142. Then, in the dispensing device 142, the culture medium is supplied from the culture medium supply device 161 to the flask 2.

Further, when performing the cell proliferation by using a new flask unit 1, the cell proliferation is performed, for example, by the following procedure.

That is, the first transport robot 171 transports the flask unit 1, which is not filled with the culture solution, on the rack 112 into the liquid operation area R4 via the stocker second door 114. The flask unit 1 is transferred to the dispensing device 142 through the third transport robot 173, and the culture medium is supplied.

Thereafter, the flask unit 1 in which the cell proliferation has progressed to finish the proliferation is transported to the dispensing device 142. Then, the culture solution in the flask 2 of the flask unit 1 in which the proliferation has been finished is dispensed into the flask 2 of the new flask unit 1. In this way, the operation of transplanting the cells into the flask 2 of the new flask unit 1 is completed. The flask unit 1 that has undergone the transplanting operation is transported to the incubator 130, and the cell proliferation is performed.

### Detailed Structure of Flask Unit

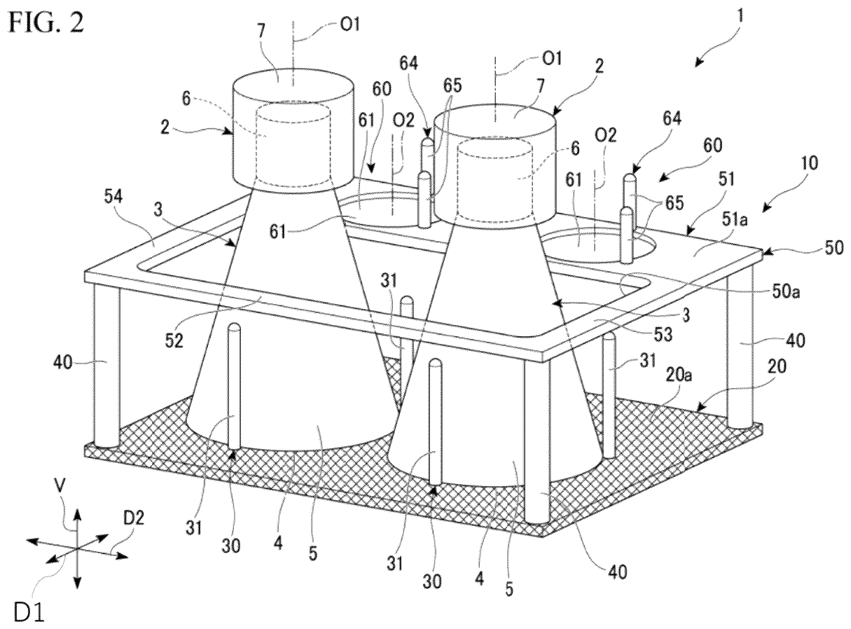

Next, a detailed structure of the flask unit 1 on which the flask 2 is mounted will be described with reference to FIGS. 2 to 6. The flask unit 1 is composed of the flask 2 and a flask tray 10.

### Flask

In the present embodiment, a flask with a lid is employed as the flask 2. The flask 2 has a flask main body 3 and a lid portion 7.

### Flask Main Body

As shown in FIGS. 2 to 5, the flask main body 3 is a so-called triangular flask. The flask main body 3 has a bottomed tubular shape centered on a flask axis O1 extending in an up-down direction V, and an upper portion thereof is open. That is, the flask main body 3 is in a posture in which a direction in which the flask axis O1 extends coincides with the up-down direction. The flask axis O1 is a central axis of the flask 2. A bottom surface 4 of the flask main body 3 forms a circular shape centered on the flask axis O1 from above when viewed in a plan view, that is, when viewed from an upper side that is one side in the up-down direction V. An outer peripheral surface 5 of the flask main body 3 is provided to rise upward from an outer peripheral edge portion of the bottom surface 4. The outer peripheral surface 5 of the flask main body 3 has a conical surface shape centered on the flask axis O1 and having a diameter which decreases toward the upper side. A mouth portion 6 that is connected to an upper end of the outer peripheral surface 5 of the flask 2 and has a cylindrical shape centered on the flask axis O1 is provided at an upper portion of the flask main body 3. A male screw twisted around the flask axis O1 is formed on the outer peripheral surface 5 at the mouth portion 6.

### Lid Portion

The lid portion 7 has a closed upper end and has a bottomed tubular shape centered on the flask axis O1. The lid portion 7 has a circular outer shape in a cross section (a horizontal cross section) orthogonal to the flask axis O1. A female screw twisted around the flask axis O1 is formed on an inner peripheral surface of the lid portion 7. The female screw of the lid portion 7 is screwed to the male screw of the mouth portion 6, so that the lid portion 7 is attached to the flask main body 3 in an airtight and liquid-tight manner.

### Flask Tray

With respect to the flask 2 having the configuration described above, various operations and transportation are performed while two flasks 2 are placed as a set on the flask tray 10.

As shown in FIGS. 2 to 5, the flask tray 10 includes a placing plate 20, a fixing portion 30, a support portion 40, a frame 50, and a lid placing portion 60. The flask tray 10 is formed of, for example, a highly corrosion-resistant metal material such as stainless steel or a synthetic resin material.

### Placing Plate

The placing plate 20 is a member on which the flask 2 is placed. The placing plate 20 has a flat plate shape extending in the horizontal direction which is a direction orthogonal to the ap-down direction V. The placing plate 20 has a rectangular shape when viewed in a plan view. Hereinafter, a lateral direction (a direction in which a pair of short sides extends) of the placing plate 20 in a plan view is referred to as a first direction D1. A longitudinal direction (a direction in which a pair of long sides extends) of the placing plate 20 in a plan view is referred to as a second direction D2.

The placing plate 20 is formed of, for example, a porous plate having a large number of through-holes in the up-down direction V, such as a punching metal, a grating material, or a mesh material. The through-holes penetrate at least in the up-down direction.

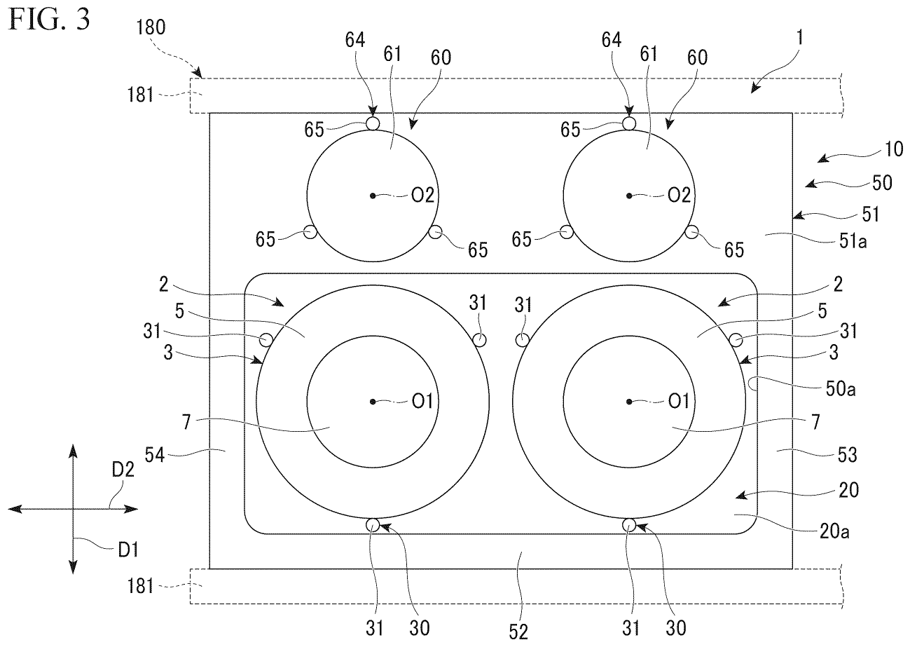

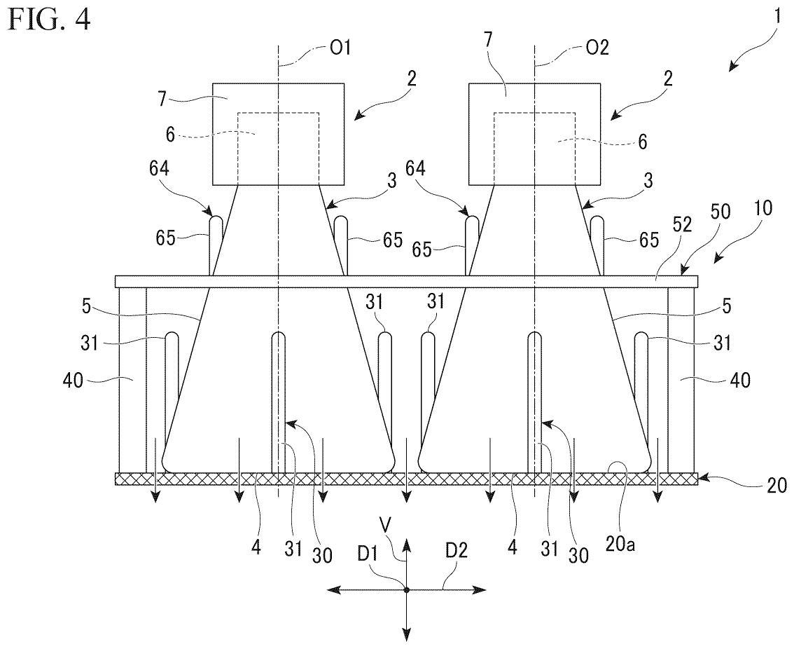

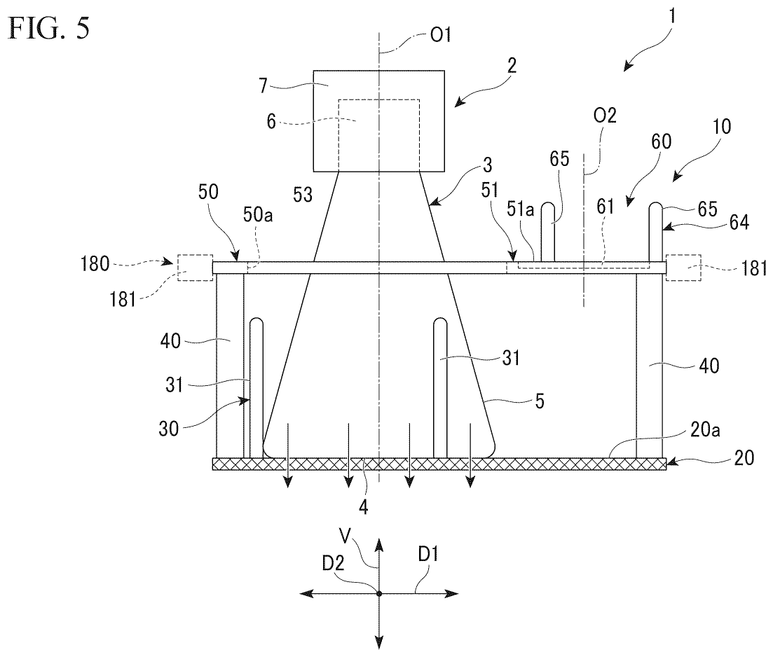

An upper surface 51a of the placing plate 20 is a placing surface 20a on which the flask 2 is placed. A plurality of (in the present embodiment, two) flasks 2 are arranged in the second direction D2 in a state where the bottom surface 4 of the flask 2 is in contact with the placing plate 20. In other words, a parallel installation direction of the plurality of flasks 2 coincides with the second direction D2. A straight line passing through the flask axes O1 of the flasks 2 in a plan view extends to coincide with the second direction D2. The plurality of flask main bodies 3 are provided at positions biased to one side (a front side of FIGS. 2 and 4, a lower side of FIG. 3, and a left side of FIG. 5) in the first direction D1 on the placing plate 20.

### Fixing Portion

As shown in FIGS. 2 to 5, the fixing portion 30 fixes the flask 2 placed on the placing surface 20a. A plurality of (in the present embodiment, two) fixing portions 30 are provided to correspond to the respective flasks 2.

The fixing portion 30 is integrally fixed to the placing surface 20a. The fixing portion 30 is configured to come into contact with the flask 2 placed on the placing surface 20a from an outer periphery side.

The fixing portion 30 of the present embodiment is composed of a plurality of fixing pins 31. Each fixing pin 31 has a lower end fixed to the placing surface 20a, and extends upward from the lower end. The plurality of fixing pins 31 are provided at intervals in a circumferential direction around the flask axis O1. In the present embodiment, three fixing pins 31 are disposed at equal intervals of 120° in the circumferential direction around the flask axis O1. The plurality of fixing pins 31 of one fixing portion 30 are provided to be in contact with a virtual circle (a virtual circle forming a maximum outer diameter of the flask 2) centered on the flask axis O1 from the outer periphery side. In this way, in a case where each flask 2 is placed on the placing surface 20a, the plurality of fixing pins 31 come into contact with the outer peripheral surface 5 of the flask 2 from the outer periphery side.

Here, the plurality of fixing pins 31 are provided to sandwich the flask axis O1 from both sides in the first direction D1. In the present embodiment, one fixing pin 31 is provided on one side of the flask axis O1 in the first direction D1. The fixing pin 31 can be brought into contact with a portion of the outer peripheral surface 5 of the flask main body 3, which is closest to one side in the first direction D1. In addition, two fixing pins 31 are provided on the other side (a side opposite to one side in the first direction D1, a back side of FIGS. 2 and 4, an upper side of FIG. 3, and a right side of FIG. 5) of the flask axis O1 in the first direction D1 to be spaced apart from each other in the second direction D2. These two fixing pins 31 can be brought into contact with portions of the outer peripheral surface 5 of the flask main body 3, which are closer to one side in the first direction D1 rather than a portion closest to the other side in the first direction D1.

An upper end of the fixing pin 31 has a hemispherical shape that is convex upward. The upper end of the fixing pin 31 may have a shape that is reduced in diameter toward the upper side, and may be, for example, a rounded shape or a needle-like shape.

### Support Portion

As shown in FIGS. 2 to 5, the support portion 40 is a rod-like member that extends upward from the placing surface 20a. A plurality of (in the present embodiment, four) support portions 40 are provided. Each support portion 40 is provided at each of four corners where a short side and a long side of the placing plate 20 in a plan view are connected to each other. An upper end of the support portion 40 is located on an upper side than the upper end of the fixing pin 31 of the fixing portion 30. The upper end of the support portion 40 is located on a lower side with respect to an upper end of the mouth portion 6 of the flask main body 3 placed on the placing surface 20a. The heights of the upper ends of the plurality of support portions 40 are the same as each other.

### Frame

As shown in FIGS. 2 to 5, the frame 50 is supported by the support portions 40 above the placing plate 20. The frame 50 has a rectangular shape in which the first direction D1 is set as a lateral direction and the second direction D2 is set as a longitudinal direction in a plan view, similarly to the placing plate 20. The contour shape of the frame 50 in a plan view is the same as that of the placing plate 20. The upper ends of the support portions 40 are connected to the lower surfaces of the four corners of the frame 50. In this way, the frame 50 is disposed above the placing plate 20 to be parallel to the placing plate 20.

An opening portion 50a that penetrates upward is formed in the frame 50. The opening portion 50a has a rectangular shape in which the first direction D1 is set as a lateral direction and the second direction D2 is set as a longitudinal direction in a plan view. The opening portion 50a extends over the second direction D2 at a position biased to one side of the frame 50 in the first direction D1.

The height of the frame 50 is set to be a height equal to or higher than the upper end of the fixing pin 31 and equal to or lower than the upper end of the mouth portion 6 of the flask main body 3, according to the height of the upper end of the support portion 40. Therefore, the flask 2 placed on the placing surface 20a penetrates the through-hole up and down. That is, the flask 2 on the placing surface 20a is surrounded by the frame 50 from the entire surrounding area in the horizontal direction.

Here, as shown in FIG. 3, in a plan view, the flask 2 is disposed within a formation range of the opening portion 50a. In the present embodiment, the plurality of flasks 2 are within a formation range of one opening portion 50a in a plan view. That is, the frame 50 and the flask 2 do not overlap each other up and down. Therefore, the up-down movement of the flask 2 is not interfered with by the frame 50, the flask 2 can be placed on the placing surface 20a from above, and the flask 2 can be separated from the flask tray 10 by pulling the flask 2 upward from the placing surface 20a.

In the present embodiment, each of the fixing portions 30 is also within the formation range of one opening portion 50a in a plan view.

Since the opening portion 50a as described above is formed in the frame 50, the frame 50 has four members such as a first frame piece 51, a second frame piece 52, a third frame piece 53, and a fourth frame piece 54.

The first frame piece 51 is a portion that extends in the second direction D2 along an outer edge of the frame 50 on the other side in the first direction D1. The second frame piece 52 is a portion that extends in the second direction D2 along an outer edge of the frame 50 on one side in the first direction D1. The third frame piece 53 is a portion that extends in the first direction D1 along an outer edge of the frame 50 on one side (a right side of FIGS. 2 and 4, a right side of FIG. 3, and a front side of FIG. 5) in the second direction D2. The second frame piece 52 is a portion that extends in the first direction D1 along an outer edge of the frame 50 on the other side (a left side of FIGS. 2 and 4, a left side of FIG. 3, and a back side of FIG. 5) in the second direction D2.

In the frame 50, the first frame piece 51, the second frame piece 52, the third frame piece 53, and the fourth frame piece 54 are connected to each other, so that the frame 50 has a frame shape surrounding the flask 2 in the horizontal direction.

Here, since the opening portion 50a of the frame 50 is formed to be biased to one side in the first direction D1, the first frame is set to have a larger dimension in the first direction D1 than the other frame pieces. The dimension in the first direction D1 in the first frame is set to be larger than the diameter of the lid portion 7. The upper surface 51a of the frame 50 is a flat surface that extends in the horizontal direction. The upper surface 51a of the frame 50 has a rectangular shape in which the first direction D1 is set as a lateral direction and the second direction D2 is set as a longitudinal direction.

### Lid Placing Portion

As shown in FIGS. 2, 3, and 5, the lid placing portion 60 is a portion that can hold the lid portion 7 removed from the flask main body 3. The lid placing portion 60 is provided on the first frame piece 51 of the frame 50. The lid placing portion 60 is disposed close to each flask 2 in the first direction D1 to correspond to each flask 2, and a plurality of the lid placing portions 60 are provided side by side in the second direction D2.

The lid placing portion 60 of the present embodiment is composed of a recessed portion 61 formed in the frame 50 and a holding portion 64.

### Recessed Portion

The recessed portion 61 is formed to be recessed downward from the upper surface 51a. The recessed portion 61 has a circular shape centered on a recessed portion axis O2 extending in a vertical direction. The recessed portion axis O2 is a central axis of the recessed portion 61. An inner diameter of the recessed portion 61 is set to be slightly larger than an outer diameter of the lid portion 7. In this way, the recessed portion 61 is configured such that the lid portion 7 is inserted therein from above and fitted thereto through a gap.

The recessed portion axis O2 of the recessed portion 61 of the lid placing portion 60 and the flask axis O1 of the flask 2 corresponding to the lid placing portion 60 are disposed on a straight line extending in the first direction D1 in a plan view. The recessed portion axes O2 of the recessed portions 61 of the plurality of lid placing portions 60 are disposed on a straight line extending in the second direction D2 in a plan view.

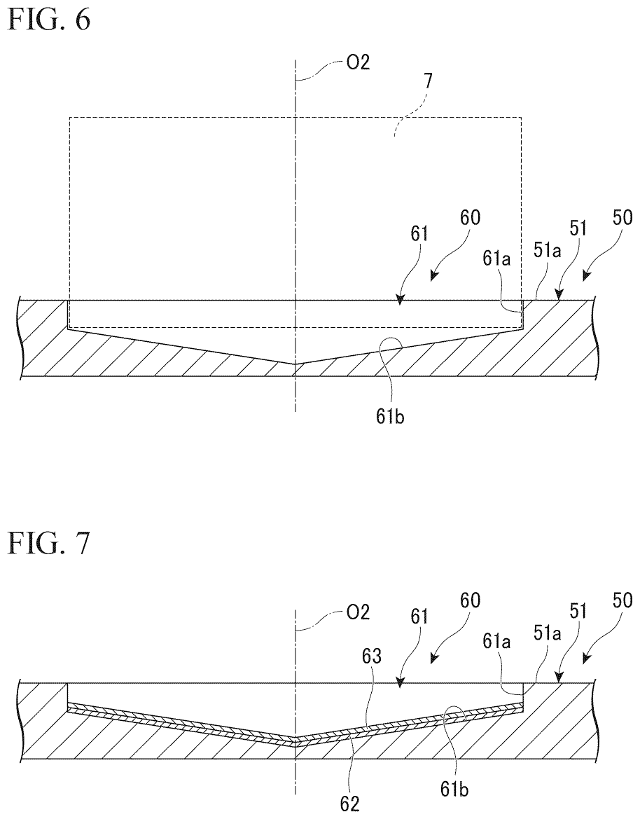

Here, as shown in FIG. 6, the recessed portion 61 has an inner periphery cylindrical surface 61a and a recessed portion bottom surface 61b. The inner periphery cylindrical surface 61a has a cylindrical surface shape centered on the recessed portion axis O2, and holds a lower portion of the lid portion 7 fitted to the recessed portion 61 from the outer periphery side. The cylindrical inner peripheral surface faces the lid portion 7 in the circumferential direction with a slight gap therebetween. The recessed portion bottom surface 61b has a tapered surface shape that decreases in diameter toward a lower side from the lower end of the inner periphery cylindrical surface 61a, and in the present embodiment, the recessed portion bottom surface 61b has a conical surface shape having an apex on the recessed portion axis O2. The lower end of the lid portion 7 is positioned in the up-down direction V by coming into contact with a boundary between the inner periphery cylindrical surface 61a and the recessed portion bottom surface 61b. The depth of the cylindrical inner peripheral surface is set to a dimension in which only a part of the lower portion of the lid portion 7 is inserted into the recessed portion 61.

### Holding Portion

As shown in FIGS. 2 to 5, the holding portion 64 is integrally provided on the upper surface 51a of the frame 50. The holding portion 64 holds the upper portion of the lid portion 7 exposed from the recessed portion 61 from the outer periphery side.

The holding portion 64 of the present embodiment is composed of a plurality of holding pins 65. Each holding pin 65 has a lower end fixed to the upper surface 51a of the frame 50, and extends upward from the lower end. The plurality of holding pins 65 are provided at intervals in the circumferential direction around the recessed portion axis O2. In the present embodiment, three holding pins 65 are disposed at equal intervals of 120° in the circumferential direction around the recessed portion axis O2. The plurality of holding pins 65 of one holding portion 64 are disposed to be in contact with a circular outer peripheral edge portion of the recessed portion 61 in a plan view.

Here, the plurality of holding pins 65 are provided to sandwich the recessed portion axis O2 from both sides in the first direction D1. In the present embodiment, two holding pins 65 are provided on one side of the recessed portion axis O2 in the first direction D1 to be spaced from each other in the second direction D2. These two holding pins 65 are disposed at portions closer to the other side in the first direction D1 rather than a portion closest to one side in the first direction D1 in the outer peripheral edge portion of the recessed portion 61. In addition, one holding pin 65 is provided on the other side of the recessed portion axis O2 in the first direction D1. The holding pin 65 is disposed at a portion of the outer peripheral edge portion of the recessed portion 61, which is closest to the other side in the first direction D1.

The upper end of the holding pin 65 has a hemispherical shape that is convex toward the upper side. The upper end of the bolding pin 65 may have a shape that is reduced in diameter toward the upper side, and may be, for example, a rounded shape or a needle-like shape. The height of the upper end of the holding pin 65 is set to a height equal to or lower than the upper end of the mouth portion 6 of the flask main body 3 placed on the placing surface 20a.

### Operation and Effects

The flask 2 is transported as the flask unit 1 by the transport device 170 in a state of being placed on the flask tray 10. Then, various dispensing operations are performed on the flash 2 at the transport destination.

When the flask unit 1 is transported by the transport device 170, the flask unit 1 is gripped by a pair of grip pieces 181 of a gripper 180 of each of the first transport robot 171, the second transport robot 172, the third transport robot 173, and the fourth transport robot 174.

That is, for example, as shown in FIGS. 3 and 5, the pair of grip pieces 181 of the gripper 180 grips the flask unit 1 to sandwich the outer peripheral edge portions of the first frame piece 51 and the second frame piece 52 in the frame 50 of the flask unit 1 therebetween. By gripping the flask unit 1 from both sides in the first direction D1 in this manner, the flask unit 1 can be stably transported even in a case where the center of gravity of the flask unit 1 is not at the center of the flask unit 1. Then, the flask unit 1 is transported to a target location by moving and turning each transport robot on the rail. In the flask unit 1, the first direction D1, which is a turning direction of the transport robot, is a main transport direction, and an inertial force in the first direction D1 is applied thereto.

Here, for example, in a case where a culture medium is supplied into the flask 2, the flask unit 1 is transported into the dispensing device 142 by the third transport robot 173. In the flask unit 1 that has reached the culture medium supply location in the dispensing device 142, the lid portion 7 is removed from the flask 2 by a lid attachment/detachment device (not shown). That is, the lid attachment/detachment device separates the lid portion 7 from the flask main body 3 by rotating the lid around the flask axis O1 in a state where the lid portion 7 is gripped. Then, the removed lid portion 7 is transported to the other side in the first direction D1 and is made to reach directly above the lid placing portion 60 of the flask tray 10. Thereafter, the lid attachment/detachment device transports the lid portion 7 downward, so that the lid portion 7 is inserted into the recessed portion 61 of the lid placing portion 60 and is fitted thereto. In this way, the lid portion 7 is placed on the lid placing portion 60 to correspond to the flask main body 3 on a one-to-one basis.

Then, a culture medium is supplied into the flask main body 3 from which the lid portion 7 is removed, by a culture medium supply nozzle (not shown). After the culture medium is supplied into the flask main body 3, the flask main body 3 is transported from the culture medium supply location together with the flask unit 1 in a state where the lid portion 7 is removed. Then, the lid portion 7 is attached to the flask main body 3 by the lid attachment/detachment device at a location different from the culture medium supply location.

As described above, in the present embodiment, after the dispensing operation is performed on the flask main body 3, the entire flask unit 1 can be transported to the next step in a state where both the flask main body 3 and the lid portion 7 are placed on the same flask tray 10. Therefore, the flask unit 1 can be moved from the dispensing operation location without performing the mounting operation of the lid portion 7 on the spot. Therefore, the other flask unit 1 on which the dispensing operation is to be performed next can be quickly transported to the dispensing operation location without waiting for the time required for the attachment of the lid portion 7 of the flask unit 1 on which the dispensing operation has been already performed. Therefore, it is possible to improve the efficiency of the dispensing operation to a large number of flask units 1.

In addition, the lid portion 7 removed from the flask main body 3 is transported while being placed on the lid placing portion 60 close to the flask main body 3 together with the corresponding flask main body 3. Therefore, the correspondence relationship between the flask main body 3 and the lid portion 7 is not broken.

In a case where the lid portion 7 is removed from the flask main body 3 and the lid portion 7 is placed in the dispensing device, when the flask main body 3 is moved, a correspondence relationship between the flask main body 3 and the lid portion 7 may be broken, and as a result, the lid portion 7 may be attached to another flask main body 3, resulting in occurrence of contamination.

On the other hand, in the present embodiment, since the entire flask unit 1 can be transported without breaking the correspondence relationship with the flask main body 3 even in a case where the lid portion 7 is removed, the lid portion 7 is not misplaced. Accordingly, it is possible to avoid the occurrence of contamination.

In addition, in the present embodiment, since two flasks 2 can be transported by one flask tray 10, the transport efficiency and the treatment efficiency can be improved. Further, since each lid placing portion 60 is provided close to the placing location of each flask 2 in the flask tray 10, the correspondence relationship between the flask main body 3 and the lid portion 7 can be maintained. Accordingly, it is possible to avoid confusion of the lid portions 7 of the flasks 2 placed on the same flask tray 10, and it is possible to further suppress the occurrence of contamination.

Furthermore, in the present embodiment, since the fixing portion 30 is provided on the placing surface 20a of the placing plate 20, a tall flask 2 can be stably placed on the placing surface 20a.

Since the fixing portion 30 is composed of the plurality of fixing pins 31 and the fixing pins 31 come into contact with the outer peripheral surface 5 of the flask main body 3, the flask 2 can be appropriately fixed without applying an excessive restraining Force to the flask main body 3.

In addition, the flask 2 can be fixed only by performing an operation of inserting the flask 2 through the opening portion 50a of the frame 50 in order to place the flask 2 on the placing surface 20a. In this case, since the upper end of the fixing pin 31 has a shape having a diameter decreasing toward the upper side, even in a case where the relative position between the flask 2 and the flask tray 10 is slightly deviated in the horizontal direction, the upper end of the fixing pin 31 guides the horizontal position of the flask 2 to an appropriate position. Accordingly, the placing work of the flask 2 can be performed more smoothly.

In addition, the height of the fixing pin 31 is set to be lower than the height of the frame 50. In this way, the fixing pin 31 does not hinder the work of attaching and detaching the lid portion 7, and the work can be smoothly performed. Furthermore, even in a case where a large inertial force is applied to the flask 2 and the fixation of the flask main body 3 by the fixing portion 30 is released, the flask 2 is held by each frame piece of the frame 50 provided above the fixing pin 31. In this way, it is possible to avoid the fall of the flask 2.

Further, the plurality of fixing pins 31 come into contact with the outer peripheral surface 5 of the flask main body 3 from both sides in the first direction D1. Therefore, the flask 2 can be held from both sides of the flask unit 1 in a traveling direction. Therefore, it is possible to avoid the fall of the flask 2 due to an inertial force when the flask tray 10 is moved.

In addition, each of the recessed portion 61 and the holding portion 64 configuring the lid placing portion 60 is located on a lower side with respect to the upper end of the mouth portion 6 of the flask 2. Therefore, it is possible to shorten the movement trajectory of the lid portion 7 after the lid portion 7 is removed from the flask main body 3. That is, in a case where the lid placing portion 60 is located on an upper side with respect to the mouth portion 6, the lid portion 7 is placed on the lid placing portion 60 by three operations of raising the lid portion 7 once, then moving the lid portion 7 in the horizontal direction, and then lowering the lid portion 7. On the other hand, in the present embodiment, since two operations of moving the lid portion 7 in the horizontal direction and then lowering the lid portion 7 are sufficient, the lid portion 7 can be efficiently removed and placed.

In this way, the lid portion 7 removed from the flask 2 can be smoothly moved to the lid placing portion 60. In addition, even in a case where the flask 2 is likely to fall, the fid placing portion 60 abuts against the flask 2, so that the flask 2 can be prevented from falling.

Further, even in a case where the lid portion 7 is placed on the lid placing portion 60 in a case where the culture solution is attached to the lid portion 7, the culture solution dropped from the lid portion 7 is collected at the center of the recessed portion 61 according to the tapered shape of the bottom surface 4 of the recessed portion 61. In this way, it is possible to avoid the scattering or the like of the culture solution from the recessed portion 61. Therefore, it is possible to further improve hygiene and to avoid contamination.

In addition, since the lid placing portion 60 includes the holding portion 64 that holds the lid portion 7 inserted into the recessed portion 61 from the outer periphery side, the lid portion 7 can be stably held.

Further, since the holding portion 64 is composed of the plurality of holding pins 65, the lid portion 7 can be appropriately held without applying an excessive restraining force to the lid portion 7. Since the upper end of the holding pin 65 is located on a lower side with respect to the mouth portion 6, the lid portion 7 removed from the flask main body 3 can be smoothly moved to the lid placing portion 60. In addition, since it is possible to avoid the size of the flask tray 10 from becoming unnecessarily large beyond the size of the flask 2, it is possible to maintain the transportability and the operability of the flask unit 1.

In addition, since the upper end of the holding pin 65 has a reduced diameter like the fixing pin 31, even in a case where the lid portion 7 is misaligned in the horizontal direction with respect to the recessed portion 61, the lid portion 7 can be smoothly inserted into the recessed portion 61 by guiding the lid portion 7 by the holding pin 65.

Further, since the holding pin 65 is disposed to come into contact with the lid portion 7 from both sides in the first direction D1 as in the case of the fixing pin 31, the lid portion 7 can be reliably held against the inertial force during the transport of the flask unit 1.

Here, in a case where the flask unit 1 is located in the clean bench 140, the flask unit 1 is exposed to the descending air current in the clean bench 140. In this case, the descending air current flows to pass through the placing plate 20.

In a case where the placing plate 20 is a flat plate having no pores, the descending air current is turned by colliding with the placing plate 20, and in this the cells, the culture medium, and other impurities are entrained.

On the other hand, in the present embodiment, since the placing plate 20 is formed of a porous plate, the descending air current passes through the placing plate 20 up and down. In particular, in a case where there is a space below the placing plate 20 at the placing location, the descending air current more appropriately passes through the placing plate 20. In this way, since the descending air current can be prevented from being turned on the placing plate 20, it is possible to prevent impurities from being rolled up on the placing plate 20. In this way, it is possible to ensure hygiene and avoid contamination.

### Second Embodiment

Next, a second embodiment of the present disclosure will be described with reference to FIG. 7. In the second embodiment, the same components as those of the first embodiment are denoted by the same reference numerals and the detailed description thereof will be omitted.

In the second embodiment, the configuration of the lid placing portion 60 is different from that in the first embodiment. That is, the lid placing portion 60 of the second embodiment has a porous member 62 and a photocatalyst layer 63, in addition to the configuration of the first embodiment.

The porous member 62 is provided at the bottom surface 4 of the lid placing portion 60. The porous member 62 has a shape along the tapered shape of the bottom surface 4 and is laminated on the surface of the lid placing portion 60 with a predetermined thickness. The porous member 62 is a member in which a large number of fine hole portions are formed, and is formed of a porous material such as diatomite or zeolite, for example.

The photocatalyst layer 63 is provided to be laminated on the entire surface of the porous member 62. The photocatalyst layer 63 contains a photocatalyst such as titanium dioxide, for example. The photocatalyst layer 63 may be formed in a sheet shape in which a large number of holes are formed, or may be configured to be applied onto the porous member 62 through spraying.

According to the second embodiment as described above, the photocatalyst layer 63 is irradiated with ultraviolet rays by an ultraviolet irradiation device provided at any location of the cell culture system 100, and thus the recessed portion 61 of the lid placing portion 60 can be sterilized. Therefore, by regularly transporting the flask wait 1 to the ultraviolet irradiation device by the transport device 170, the hygiene of the flask unit 1 can be ensured.

In addition, since the porous member 62 is provided at the bottom surface 4 of the lid placing portion 60, the culture solution adhered to the lid portion 7 is absorbed by the porous member 62 and collected at the center of the recessed portion 61. In this way, it is possible to more reliably prevent the culture solution from scattering, and it is possible to avoid contamination.

### Third Embodiment

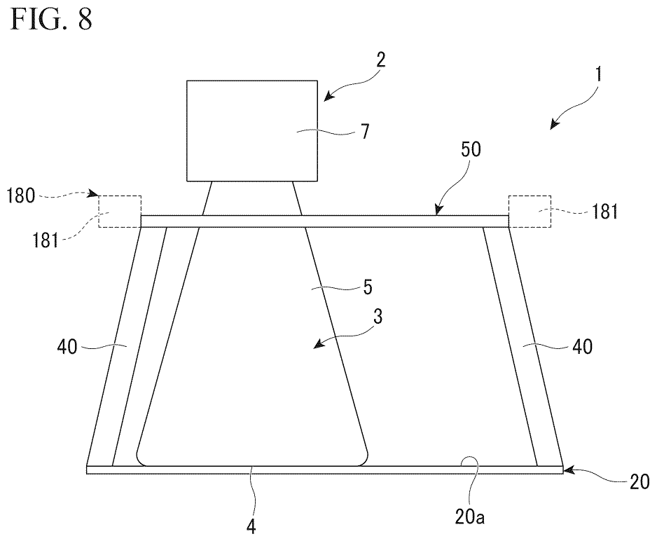

Next, a second embodiment of the present disclosure will be described with reference to FIG. 8. In the third embodiment, the same components as those of the first embodiment are denoted by the same reference numerals and a detailed description thereof will be omitted.

In the third embodiment, the shapes of the placing plate 20 and the frame 50 are different from those in the first embodiment. That is, in the third embodiment, the dimension of the frame 50 in the first direction D1 is shorter than the dimension of the placing plate 20 in the first direction D1. Accordingly, the frame 50 is disposed above the placing plate 20 and inside in the first direction D1 when viewed in the second direction D2. In addition, along with this, the pair of support portions 40 disposed to be spaced apart from each other in the first direction D1 extend to be brought closer to each other toward the upper side. Accordingly, the flask tray 10 is configured to have a trapezoidal contour when viewed in the second direction D2.

In the third embodiment, with the above configuration, the access space for the grip piece 181 of the gripper 180 can be increased at the upper portion of the flask tray 10. That is, since the flask tray 10 has a trapezoidal shape in which an upper bottom is shorter than a lower bottom when viewed in the second direction D2, a configuration is made in which the upper part is compact. Therefore, a large space can be provided at the corner portion of the upper portion of the flask tray 10.

Accordingly, the gripability of the gripper 180 can be improved, and the transportability of the flask unit 1 can be further improved.

### Fourth Embodiment

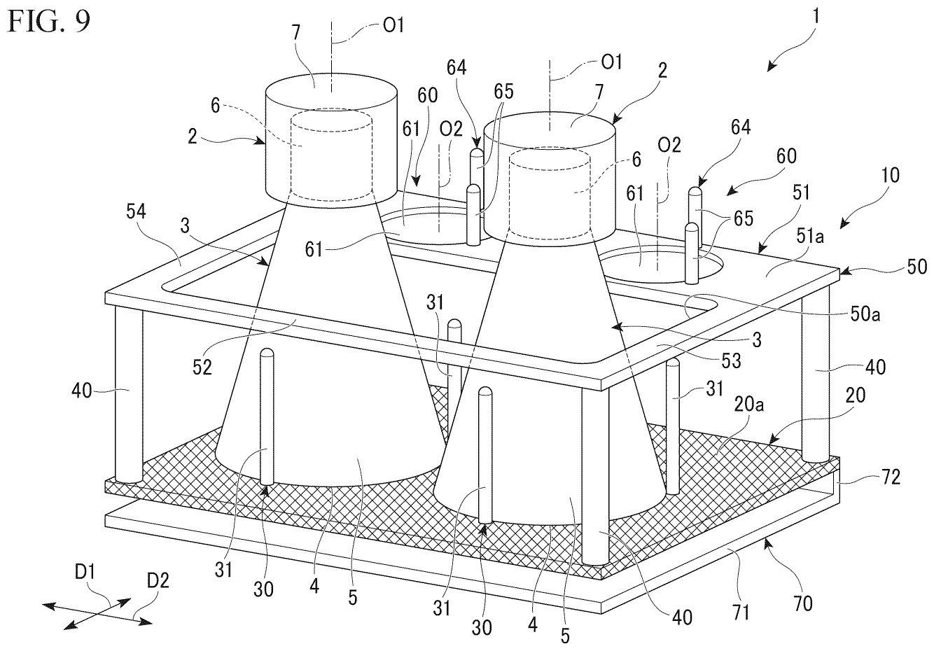

Next, a second embodiment of the present disclosure will be described with reference to FIG. 9. In the fourth embodiment, the same components as those of the other embodiments are denoted by the same reference numerals and a detailed description thereof will be omitted.

The flask tray 10 of the flask unit 1 of the present embodiment has a leg portion 70 in addition to the configuration of the first embodiment.

### Leg Portion

As shown in FIG. 9, the leg portion 70 extends downward from the placing plate 20 and supports the placing plate 20. The leg portion 70 of the present embodiment has a base plate 71 and a connection plate 72.

The base plate 71 is a flat plate-shaped member extending in the horizontal direction. The base plate 71 is provided below the placing plate 20 with a gap therefrom to be parallel to the placing plate 20. The base plate 71 has a rectangular shape in which the first direction D1 is set as a lateral direction and the second direction D2 is set as a longitudinal direction in a plan view. The base plate 71 bas the same contour shape as the placing plate 20 and the frame 50 in a plan view.

The connection plate 72 is connected to the placing plate 20 in the up-down direction V. The connection plate 72 supports the placing plate 20 above the base plate 71. The connection plate 72 extends in the second direction D2 along the outer peripheral edge portions of the placing plate 20 and the base plate 71 on the other side in the first direction D1. A lower end of the connection plate 72 is connected to the base plate 71 over the entire region in the second direction D2. An upper end of the connection plate 72 is connected to the placing plate 20 over the entire region in the second direction D2. In this way, a space between the placing plate 20 and the base plate is closed by the connection plate 72 only on the other side in the first direction D1. The space between the placing plate 20 and the base plate is open in the horizontal direction on one side in the first direction D1 and on one side and the other sides in the second direction D2.

### Operation and Effects

In the present embodiment, since there is the leg portion 70 that supports the placing plate 20 from below, a space can be formed below the placing plate 20. Therefore, the descending air current can smoothly flow to pass through the placing plate 20 even without the need for devising such as forming a space below the placing location of the flask unit 1. Therefore, it is possible to further avoid the rolling-up of impurities.

In addition, in the present embodiment, the leg portion 70 is composed of the base plate 71 and the connection plate 72, and a part of the space below the placing plate 20 is closed by the connection plate 72. Therefore, it is possible to control the traveling direction of the descending air current that passes through the placing plate 20. Accordingly, it is possible to more appropriately manage the hygiene in the clean bench 140.

### Other Embodiments

A described above, each of the embodiments of the present disclosure has been described in detail with reference to the drawings. However, the specific configuration is not limited to this embodiment, and also includes design changes and the like without departing from the gist of the present disclosure.

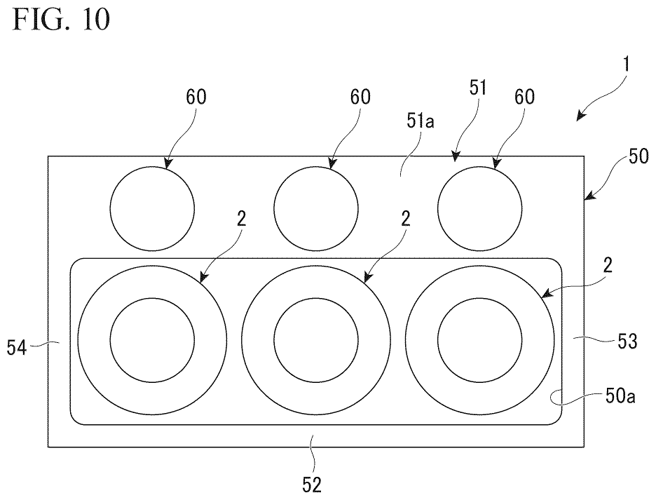

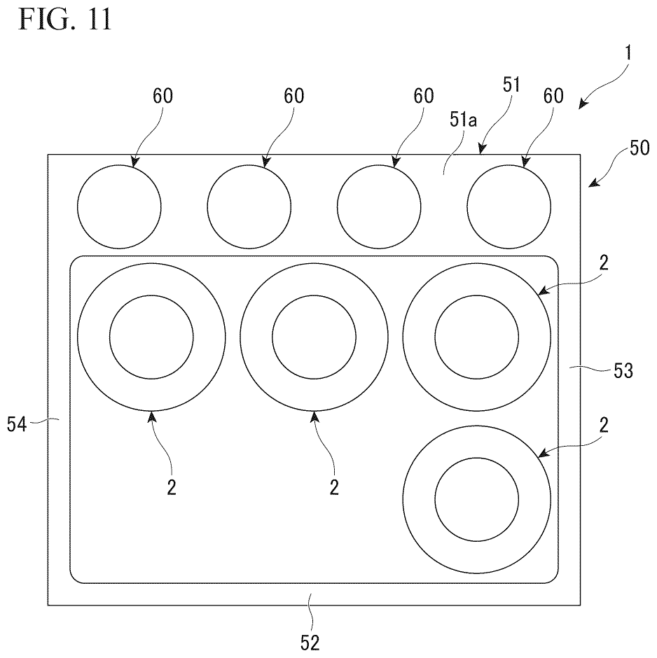

Although an example in which two flasks 2 are mounted on the flask unit 1 of the embodiment has been described, there is no limitation thereto. For example, as in a first modification example shown in FIG. 10, a configuration may be adopted in which three flasks 2 can be placed in the second direction D2 and the lid placing portions 60 are provided on one side in the first direction D1 to correspond to the respective flasks 2. In addition, the number of the flasks 2 is not limited to three, and four or more flasks 2 may be provided side by side in the second direction D2. In addition, the flask unit 1 may have only one flask 2.

In addition, for example, as in a second modification example shown in FIG. 11, a configuration may be adopted in which the flask unit 1 has four flasks 2. In this case, one flask 2 is not disposed side by side in the second direction D2, but is provided in parallel in the first direction D1, so that the flasks are placed in an L-shape in a plan view. Even in this case, the lid placing portion 60 may be disposed such that the relative position thereof is determined with respect to each flask 2. Even in a case of having five or more flasks 2, the flasks 2 may be disposed in an L-shape in a plan view.

In the embodiment, each of the placing plate 20, the frame 50, and the base plate 71 has a rectangular shape. However, there is no limitation thereto. Other shapes such as other polygons, circles, and ellipses may be adopted. On the other hand, it is preferable that the frame 50 has a pair of frame pieces spaced apart from each other in the first direction D1 or the second direction D2 and parallel to each other. In this way, the frame 50 can be easily gripped by the grip piece 181 of the gripper 180.

In the embodiment, the frame 50 is gripped by the grip piece 181 of the gripper 180 from both sides in the first direction D1, but may be gripped from both sides in the second direction D2.

In the embodiment, the frame 50 is gripped by the grip piece 181 of the gripper 180. However, for example, a configuration may be adopted in which the lower frame is gripped. Since the flask 2 has a structure in which the flask 2 is so tall that it penetrates the frame 50 upward and the center of gravity thereof is located on the upper side, the transport of the flask unit 1 is stabilized in a case where the frame 50 is gripped.

The support portion 40 is not limited to a rod shape and may have other shapes such as a plate shape. In addition, the number of the support portions 40 is not limited to four, and may be equal to or less than four as long as the frame 50 can be supported thereby.

The frame 50 has a configuration of surrounding the entire periphery of the flask 2, but may also have a configuration of surrounding at least a part thereof. For example, a configuration may be adopted in which the flask 2 is surrounded only from both sides in the first direction D1, and a configuration may be adopted in which the flask 2 is surrounded only from both sides in the second direction D2.

The lid placing portion 60 is not limited to a configuration in which both the recessed portion 61 and the holding portion 64 are provided, and either one of the recessed portion 61 or the holding portion 64 may be provided.

The configurations of the fixing portion 30 and the holding portion 64 are not limited to the pin-like configuration, and may have other configurations such as a ring-like configuration.

In the second embodiment, the configuration in which both the porous member 62 and the photocatalyst layer 63 are provided has been described. However, only one of them may be provided.

The bottom portion of the lid placing portion 60 is not limited to the tapered shape and may have another shape such as a flat surface.

### Additional Remark

The flask tray 10, the flask unit 1, and the cell culture system 100 described in each embodiment are understood as follows, for example.
(1) A flask tray 10 according to a first aspect includes: a placing plate 20 having a placing surface 20a on which a bottom surface 4 of a flask 2 having a lid portion 7 that is attachable to and detachable from the flask is placed; a support portion 40 that extends upward from the placing plate 20; and a frame 50 that is supported by the support portion 40 above the placing plate 20 and that surrounds the flask 2 from a horizontal direction such that the flask 2 is capable of being placed on the placing surface 20a from above, in which the frame 50 has a lid placing portion 60 that is disposed adjacent to the flask 2 and that is capable of holding the lid portion 7.

According to the configuration described above, after the dispensing operation is performed on the flask 2, both the flask main body 3 and the lid portion 7 removed from the flask main body 3 can be moved while being placed on the flask tray 10. That is, the dispensing operation can be performed on the flask 2 of the next flask unit 1 without taking time to attach the lid portion 7 on the spot. In this way, the operation efficiency can be improved.

In addition, since the flask main body 3 and the lid portion 7 are transported in a one-to-one relationship, it is possible to prevent the lid portion 7 from being attached to another flask main body 3. In this way, the occurrence of contamination can be suppressed.
(2) A flask tray 10 according to a second aspect is the flask tray 10 according to the above (1), in which the placing surface 20a is configured such that a plurality of the flasks 2 are capable of being placed side by side in a second direction D2 Intersecting a first direction D1 that is a direction in which the flask 2 and the lid placing portion 60 are adjacent to each other, and a plurality of the lid placing portions 60 are arranged in the second direction D2 to correspond to respective flasks 2.

Accordingly, since a plurality of flasks 2 can be moved by one flask tray 10, the movement efficiency and the operation efficiency can be improved. In addition, since the correspondence relationship between each flask 2 and the lid portion 7 can be maintained, it is possible to avoid mistakenly attaching the lid portion 7 to another flask 2 when the lid portion 7 is attached to the flask main body 3.
(3) A flask tray 10 according to a third aspect is the flask tray 10 according to the above (1) or (2), further including: a fixing portion 30 that is provided on the placing surface 20a and that comes into contact with the flask 2 from an outer periphery side of the flask 2.

Accordingly, the flask 2 can be stably placed on the flask tray 10.
(4) A flask tray 10 according to a fourth aspect is the flask tray 10 according to the above (3), in which the fixing portion 30 includes a plurality of fixing pins 31 that extend upward from the placing surface 20a and that are disposed in a circumferential direction of the flask 2, and an upper end portion of the fixing pin 31 is located on a lower side with respect to the frame 50 and is reduced in diameter toward an upper side.

The plurality of pins comes into contact with the outer peripheral surface 5 of the flask 2, so that the stability of the flask 2 can be ensured. In addition, even in a case where there is a slight deviation in a relative position between the flask 2 and the flask tray 10 when the flask 2 is placed on the placing surface 20a from above, the upper end of the fixing pin 31 guides the flask 2, so that the flask 2 can be placed at an appropriate position.
(5) A flask tray 10 according to a fifth aspect is the flask tray 10 according to the above (4), in which the plurality of fixing pins 31 are provided to sandwich a central axis of the flask 2 from both sides in a first direction D1 that is a direction in which the flask 2 and the lid placing portion 60 are adjacent to each other.

For example, in a case where the first direction D1 of the flask tray 10 is a traveling direction, the flask 2 can be held from both sides in the traveling direction. Therefore, it is possible to avoid the fall of the flask 2 due to an inertial force when the flask tray 10 is moved.
(6) A flask tray 10 according to a sixth aspect is the flask tray 10 according to any one of the above (1) to (5), in which the lid placing portion 60 has a recessed portion 61 that is recessed downward from an upper surface 51a of the frame 50, and into which the lid portion 7 is inserted.

The lid portion 7 is fitted into the recessed portion 61 of the lid placing portion 60, so that the lid portion 7 can be stably held.
(7) A flask tray 10 according to a seventh aspect is the flask tray 10 according to the above (6), in which a bottom surface 4 of the recessed portion 61 is a tapered surface that is tapered downward toward a center of the bottom surface 4.

Even in a case where a liquid such as a culture solution adheres to the lid portion 7, the liquid dropped from the lid portion 7 can be collected at the center of the lid placing portion 60. Accordingly, hygiene can be ensured, and contamination due to the scattering of the liquid can be avoided.
(8) A flask tray 10 according to an eighth aspect is the flask tray 10 according to the above (6) or (7), in which the lid placing portion 60 further includes a porous member 62 provided at a bottom surface 4 of the recessed portion 61.

In this way, it is possible to further avoid the scattering of the liquid dropped from the lid portion 7.
(9) A flask tray 10 according to a ninth aspect is the flask tray 10 according to any one of the above (6) to (8), in which the lid placing portion 60 further includes a photocatalyst layer 63 provided in the recessed portion 61.

Accordingly, the lid placing portion 60 can be sterilized by irradiating the lid placing portion 60 with light.
(10) A flask tray 10 according to a tenth aspect is the flask tray 10 according to any one of the above (6) to (9), in which the lid placing portion 60 further includes a holding portion 64 that is provided on an upper surface 51a of the frame 50 and that holds the lid portion 7 fitted into the recessed portion 61 from an outer periphery side.

Accordingly, the lid portion 7 can be held more stably.
(11) A flask tray 10 according to an eleventh aspect is the flask tray 10 according to the above (10), in which the holding portion 64 includes a plurality of holding pins 65 that extend upward from the upper surface 51a of the frame 50 and that are disposed in a circumferential direction of the recessed portion 61, and an upper end portion of the holding pin 65 is located on a lower side with respect to an upper end of a mouth portion 6 of the flask 2 and is reduced in diameter toward an upper side.

In this way, the lid portion 7 can be smoothly moved from the flask 2 to the lid placing portion 60, and it is possible to avoid the size of the flask tray 10 from being unnecessarily increased beyond the size of the flask 2.
(12) A flask tray 10 according to a twelfth aspect is the flask tray 10 according to the above (11), in which the plurality of holding pins 65 are provided to sandwich a central axis of the recessed portion 61 from both sides in a first direction D1 that is a direction in which the flask 2 and the lid placing portion 60 are adjacent to each other.

For example, in a case where the first direction D1 of the flask tray 10 is a traveling direction, the lid portion 7 can be held from both sides in the traveling direction. Therefore, it is possible to avoid the fall of the flask 2 due to an inertial force when the flask tray 10 is moved.
(13) A flask tray 10 according to a thirteenth aspect is the flask tray 10 according to any one of the above (1) to (12), in which the placing plate 20 is formed of a porous plate having a plurality of holes.

In this way, in a case where the flask tray 10 is exposed to the descending air current, the descending air current passes through the placing plate 20 as it is. Therefore, it is possible to avoid the recent or impurities from being rolled up due to the deflection of the air current or the like on the placing surface 20a of the placing plate 20.
(14) A flask tray 10 according to a fourteenth aspect is the flask tray 10 according to the above (13), further including a leg portion 70 that extends downward from the placing plate 20 and that supports the placing plate 20.

Accordingly, a space can be formed below the placing plate 20. Therefore, the descending air current smoothly passes through the placing plate 20, and the rolling-up of air can be suppressed.
(15) A flask tray 10 according to a fifteenth aspect is the flask tray 10 according to the above (14), in which the leg portion 70 includes a base plate 71 that is provided below the placing plate 20 with a gap from the placing plate 20, and a connection plate 72 that extends along a part of an outer peripheral edge portion of the placing plate 20 and connects the placing plate 20 and the base plate 71 up and down.

In this way, it is possible to control a direction in which a descending air current that passes through the placing surface 20a flows out on a lower side of the placing surface 20g.
(16) A flask tray 10 according to a sixteenth aspect is the flask tray 10 according to any one of the above (1) to (15), in which a dimension of the frame 50 in the horizontal direction is smaller than a dimension of the placing plate 20 in the horizontal direction when viewed in the horizontal direction.

Accordingly, for example, when the frame 50 is held by the gripper 180, an access space for the gripper 180 can be secured to be wide.
(17) A flask unit 1 according to a seventeenth aspect includes: the flask tray 10 according to any one of the above (1) to (16); and the flask 2 having the lid portion 7.

In this way, it is possible to improve the movement efficiency and the operation efficiency.
(18) The flask unit 1 according to an eighteenth aspect is the flask unit 1 according to the above (17), in which a position in an up-down direction V of the lid placing portion 60 is at a height equal to or lower than an upper end of a mouth portion 6 of the flask 2.

In this way, the lid portion 7 removed from the flask 2 can be smoothly moved to the lid placing portion 60.
(19) A cell culture system 100 according to a nineteenth aspect includes: the flask unit 1 according to the above (17) or (18); an incubator 130 that forms a culture area R3 in which the flask unit 1 is accommodated; a clean bench 140 that forms a liquid operation area R4 for performing a dispensing operation on the flask unit 1; a transport device 170 that is capable of transporting the flask unit 1 in each of the culture area R3 and the liquid operation area R4; and a pass box 150 that is capable of transferring the flask unit 1 between the incubator 130 and the clean bench 140.

In the cell culture system 100 as described above, the flask unit 1 described above is used, so that it is possible to improve the culture efficiency.

### INDUSTRIAL APPLICABILITY

According to the flask tray, the flask unit, and the cell culture system of the present disclosure, it is possible to improve treatment efficiency and to suppress occurrence of contamination.

### REFERENCE SIGNS LIST

1 Flask unit
2 Flask
3 Flask main body
4 Bottom surface
5 Outer peripheral surface
6 Mouth portion
7 Lid portion
10 Flask tray
20 Placing plate
20a Placing surface
30 Fixing portion
31 Fixing pin
40 Support portion
50 Frame
50a Opening portion
51 First frame piece
51a Upper surface
52 Second frame piece
53 Third frame piece
54 Fourth frame piece
60 Lid placing portion
61 Recessed portion
61 a Inner periphery cylindrical surface
61b Recessed portion bottom surface
62 Porous member
63 Photocatalyst layer
64 Holding portion
65 Holding pin
70 Leg portion
71 Base plate
72 Connection plate
100 Cell culture system
110 Stocker
111 Stocker main body
112 Rack
113 Stocker first door
114 Stocker second door
120 Loading booth
121 Booth main body
122 Loading door
130 Incubator
131 Incubator main body
132 Shaking stage
140 Clean bench
141 Clean bench main body
142 Dispensing device
145 Waste liquid extracting device
149 Stage
150 Pass box
151 Box main body
152 First door
153 Second door
160 Measurement device
161 Culture medium supply device
162 Waste liquid tank
170 Transport device
171: First transport robot
172 Second transport robot
173 Third transport robot
174 Fourth transport robot
175 Conveyor
180 Gripper
181 Grip piece
R1 Storage area
R2 Loading area
R3 Culture area
R4 Liquid operation area
D1 First direction
D2 Second direction
O1 Flask axis
O2 Recessed portion axis

1. A flask tray comprising:
a placing plate having a placing surface on which a bottom surface of a flask having a lid portion that is attachable to and detachable from the flask is placed;
a support portion that extends upward from the placing plate; and
a frame that is supported by the support portion above the placing plate and that surrounds the flask from a horizontal direction such that the flask is placed on the placing surface from above,
wherein the frame has a lid placing portion that is disposed adjacent to the flask and that holds the lid portion.

2. The flask tray according to claim 1,
wherein the placing surface is configured such that a plurality of the flasks are placed side by side in a second direction intersecting a first direction that is a direction in which the flask and the lid placing portion are adjacent to each other, and
a plurality of the lid placing portions are arranged in the second direction to correspond to respective flasks.

3. The flask tray according to claim 1, further comprising:
a fixing portion that is provided on the placing surface and that comes into contact with the flask from an outer periphery side of the flask.

4. The flask tray according to claim 3,
wherein the fixing portion includes a plurality of fixing pins that extend upward from the placing surface and that are disposed in a circumferential direction of the flask, and
an upper end portion of the fixing pin is located on a lower side with respect to the frame and is reduced in diameter toward an upper side.

5. The flask tray according to claim 4,
wherein the plurality of fixing pins are provided to sandwich a central axis of the flask from both sides in a first direction that is a direction in which the flask and the lid placing portion are adjacent to each other.

6. The flask tray according to claim 1,
wherein the lid placing portion has a recessed portion that is recessed downward from an upper surface of the frame, and into which the lid portion is inserted.

7. The flask tray according to claim 6,
wherein a bottom surface of the recessed portion is a tapered surface that is tapered downward toward a center of the bottom surface.

8. The flask tray according to claim 6,
wherein the lid placing portion further includes a porous member provided at a bottom surface of the recessed portion.

9. The flask tray according to claim 6,
wherein the lid placing portion further includes a photocatalyst layer provided in the recessed portion.

10. The flask tray according to claim 6,
wherein the lid placing portion further includes a holding portion that is provided on an upper surface of the frame and that holds the lid portion fitted into the recessed portion from an outer periphery side.

11. The flask tray according to claim 10,
wherein the holding portion includes a plurality of holding pins that extend upward from the upper surface of the frame and that are disposed in a circumferential direction of the recessed portion, and
an upper end portion of the holding pin is located on a lower side with respect to an upper end of a mouth portion of the flask and is reduced in diameter toward an upper side.

12. The flask tray according to claim 11,
wherein the plurality of holding pins are provided to sandwich a central axis of the recessed portion from both sides in a first direction that is a direction in which the flask and the lid placing portion are adjacent to each other.

13. The flask tray according to claim 1,
wherein the placing plate is formed of a porous plate having a plurality of holes.

14. The flask tray according to claim 13, further comprising:
a leg portion that extends downward from the placing plate and supports the placing plate.

15. The flask tray according to claim 14,
wherein the leg portion includes
a base plate that is provided below the placing plate with a gap from the placing plate, and
a connection plate that extends along a part of an outer peripheral edge portion of the placing plate and connects the placing plate and the base plate up and down.

16. The flask tray according to claim 1,
wherein a dimension of the frame in the horizontal direction is smaller than a dimension of the placing plate in the horizontal direction when viewed in the horizontal direction.

17. A flask unit comprising:
the flask tray according to claim 1; and
the flask having the lid portion.

18. The flask unit according to claim 17,
wherein a position in an up-down direction of the lid placing portion is at a height equal to or lower than an upper end of a mouth portion of the flask.

19. A cell culture system comprising:
the flask unit according to claim 17;
an incubator that forms a culture area in which the flask unit is accommodated;
a clean bench that forms a liquid operation area for performing a dispensing operation on the flask unit;
a transport device that transports the flask unit in each of the culture area and the liquid operation area; and
a pass box that transfers the flask unit between the incubator and the clean bench.
